(19) United States
(12) Patent Application Publication
(10) Pub. No.: US 2008/0286146 A1
(43) Pub. Date: Nov. 20, 2008

(54) Cooling means with a cooling space

(75) Inventors: Katrin Schroll, Matzing (DE); Dirk Wittenberg, Munchen (DE)

Correspondence Address: OSRAM SYLVANIA INC, 100 ENDICOTT STREET, DANVERS, MA, 01923, US

(73) Assignee: PATENT-TREUHAND-GESELLSCHAFT FUR ELEKTRISCHE GLUHLAMPEN MBH, MUNCHEN (DE)

(21) Appl. No.: 12/068,592

(22) Filed: Feb. 8, 2008

(30) Foreign Application Priority Data
May 15, 2007 (DE) 10 2007 022 747.9

(51) Int. Cl. A61L 2/10

(52) U.S. Cl. 422/24

(57) ABSTRACT

A cooling apparatus with a cooling space which is especially suited for storing perishable foods is given. In the cooling apparatus there is a UV radiation source which is suited to emitting electromagnetic radiation from the ultraviolet region, and in its operation the cooling space is irradiated with this radiation. Via the electromagnetic radiation it is fundamentally possible to kill microorganisms such as bacteria and molds and thus to disinfect the refrigerator and the free surfaces contained in the cooling space as well as the air of the cooling space.

The invention relates to a cooling means with a cooling space, especially a cooling means which is suited for storing perishable foods.

For example, cooling means in the form of conventional refrigerators for private households are known. With these refrigerators it is possible to store foods at temperatures of less than or equal to roughly 7° C. At these low temperatures many bacteria and molds grow much more slowly than at conventional room temperatures so that the foods spoil much more slowly.

Reduced growth at such low temperatures however does not apply to all germs. There are microorganisms, for example so-called psychrophils, which even grow especially well at refrigerator temperatures. Moreover the growth of bacteria and molds can only be slowed down in this way, but not completely prevented. Therefore refrigerators should be regularly cleaned.

Furthermore refrigerators are known which have special coatings with silver compounds in the interior. These special coatings allegedly provide for at least partial disinfection of the refrigerator. In any case it is questionable from the current standpoint whether or to what extent this action does in fact exist since it could not be unequivocally demonstrated by various agencies. Moreover it does not yet seem to have been completely clarified yet whether and to what extent escaping silver ions can entail a health hazard.

Furthermore systems are known for releasing chlorine dioxide. They are intended to be placed in the cooling spaces of cooling devices in order to provide for disinfection of the cooling space and the things located in it by means of chlorine dioxide.

These systems are sold in the form of plastic tubes in which two different liquids are contained separately from one another. To activate the system the tube is bent so that the two liquids are mixed and chlorine dioxide is formed. The chlorine dioxide escapes via the plastic jacket. It is released for an interval of roughly 1 month starting from activation of the system. Afterwards it must be replaced.

The object is to devise a cooling means for which the amount of bacteria and mold located in the cooling space can be kept low in a technically simple manner and with low cost.

A cooling means is devised in which there is a UV radiation source which is suited to emitting electromagnetic radiation from the ultraviolet region. In operation of the UV radiation source the cooling space is irradiated by means of electromagnetic radiation.

The emission spectrum of the UV radiation source can fundamentally also contain portions which lie outside the ultraviolet region. For example, in addition to the UV portions, also other portions can be contained which lie in the wavelength range which is visible to the human eye. In one advantageous embodiment however at least a large part of the radiation intensity emitted by the UV radiation source is from the ultraviolet range. Preferably all the electromagnetic radiation is within the ultraviolet region.

UV radiation with great wavelengths is already able to destroy the chemical bonds of organic molecules. Therefore it is fundamentally possible to destroy at least some of the germs and microorganisms such as bacteria and molds contained in the refrigerator by means of UV radiation.

The use of chemicals or special coatings with silver compounds can be advantageously abandoned when using UV radiation. Thus allergies or oversensitivities to possible contents cannot arise. The system is environmentally safe since for example it is not necessary to dispose of disinfection solutions in waste water. Foods which have been irradiated with UV radiation remain neutral in taste and smell and fundamentally unburdened.

The indicated approach is fundamentally suited for all possible cooling means, such as for example cold-storage lockers or freezers. According to one preferred embodiment the cooling means is also cold storage or a mobile cooler box.

According to another embodiment the UV radiation source has at least one LED component which is suited to emitting the electromagnetic radiation. Due to the small extensions of LED components they can be specifically attached to desired locations of the cooling means with little effort.

The UV radiation source advantageously has several LED components. Several luminescent diode chips or LED components with at least one luminescent diode chip can be clustered according to another embodiment or can be distributed with a distance to one another.

Until a few years ago the radiation intensity of UV luminescent diode chips was relatively weak. Radical developments in LED technology have made the luminescent diode chip however much more efficient so that the radiation intensity of currently available UV luminescent diode chips is fundamentally suitable for efficient disinfection of cooling spaces.

By using compact LED technology generally a significant change in the design of conventional cooling means is generally not necessary. Moreover the power consumption of UV radiation-emitting LED components is relatively low. As a result of the long life of LED components the system requires very little maintenance and is cost-favorable. Due to the low temperatures which prevail in the cooling spaces in the operation of the cooling devices, the service life of the LED components is prolonged even more. In the disposal of the cooling means, due to the LED components there is no additional cost or special waste.

In addition or alternatively it is however also fundamentally possible to use other radiation sources for UV radiation. For example medium pressure or low pressure lamps, for example mercury vapor lamps, can be used. This can be advantageous especially in cooling means with especially large cooling spaces, such as for example cold storages.

According to another embodiment, the UV radiation source emits UVD radiation in its operation, i.e. electromagnetic radiation from a wavelength range of less than or equal to 300 nm. This means that at least part of the emitted electromagnetic radiation is UVD radiation. Advantageously it is also possible for all the radiation emitted by the UV radiation source to be UVD radiation.

Preferably the electromagnetic radiation contains UVC radiation, i.e. radiation from the wavelength range of less than or equal to 280 nm. The radiation can also consist especially entirely of UVC radiation. According to one development of the cooling means the electromagnetic radiation has radiation from a wavelength range between 250 nm and 270 nm inclusive or consists of this radiation. With this radiation especially efficient killing of germs is possible.

The cooling means according to another embodiment has a closing means which is suited for opening and closing an opening of the cooling space. Moreover a switching means is contained which prevents operation of the UV light source as long as the closing means is opened. Advantageously the trigger circuit for the UV light source is turned off by the switching means as long as the closing means is opened. This can prevent harmful UV radiation from reaching the eyes of a user of the cooling means. The switching means is actuated especially by means of opening and closing of the closing means.

The cooling means according to another embodiment has a trigger circuit for the UV light source which has a timer means such as for example a timer switch. Operation of the UV light source can be controlled in a specific manner by the timer means.

For example it can be set up such that the UV radiation source is automatically turned off after a certain time after closing the cooling space of the cooling means. Alternatively it can be provided for example that the UV radiation source is always continuously operated when the opening of the cooling space is closed, i.e. a timer means can also be omitted.

In another embodiment of the cooling means there is a light source with at least one LED component which is suited for emitting light which is visible to the human eye. The light source is arranged such that the cooling space is illuminated when it is in operation. According to one development the light source is electrically coupled to the UV radiation source such that the UV radiation source is turned off when the light source is operated.

In one advantageous embodiment the light source has several LED components.

Fundamentally each LED component of the light source and/or of the UV radiation source can have a single luminescent diode chip or a plurality of luminescent diode chips. The LED components according to another embodiment can also have one luminescent diode chip or can consist of it and can be free of its own component housing.

According to another embodiment, both the UV radiation source and also the light source each have one or more LED components, moreover the UV radiation source and the light source having a common circuit board on which at least some LED components of the UV radiation source and the light source are jointly mounted, electrically and mechanically. In this way the illumination for the cooling means and the device for disinfection of the cooling space can be advantageously combined with one another.

Other advantages, preferred embodiments and developments of the cooling means will become apparent from the following embodiments which are explained in conjunction with the figures.

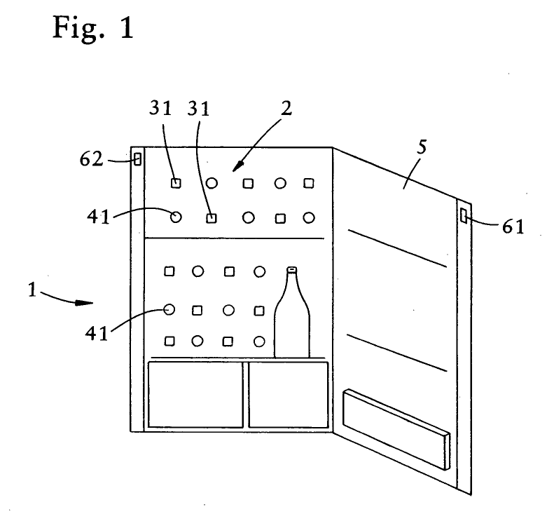

FIG. 1 shows a schematic of a cooling means according to one embodiment;

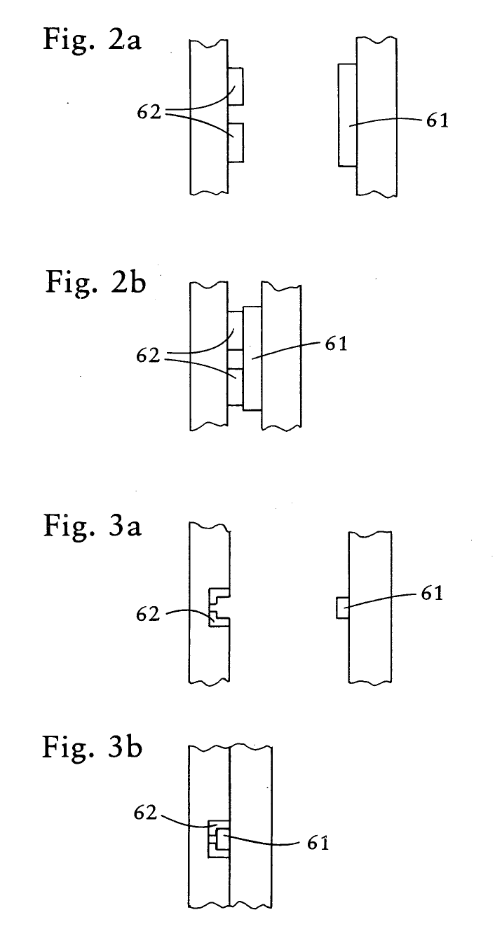

FIGS. 2a and 2b show a schematic of an extract of a cooling means with details of a switching means according to a first embodiment;

FIGS. 3a and 3b show a schematic of an extract of a cooling means with details of a switching means according to a second embodiment; and

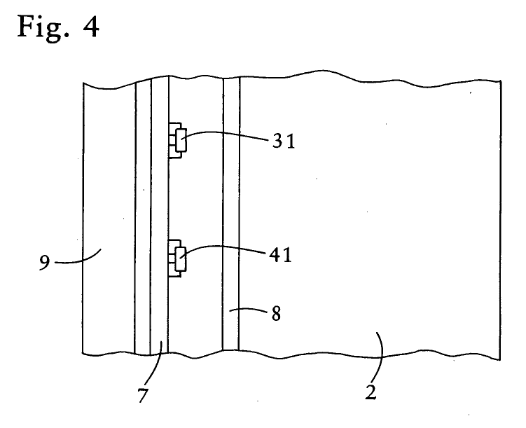

FIG. 4 shows a schematic of an extract of a cooling means with details of a UV radiation source and of a light source according to a first embodiment.

In the figures and the embodiments the same or identically acting components are each labelled with the same reference number. The illustrated components and the size ratios of the components among one another are not to scale. Rather, some details of the figures can be shown exaggerated for better understanding.

In the embodiment shown in FIG. 1 the cooling means 1 is a refrigerator for a private household. It has a cooling space 2 in which especially foods can be stored. An opening in the cooling space 2 can be closed and opened again by a closing means 5 in the form of a refrigerator door. But differently made closing means are also conceivable, such as for example hinged covers or movable closures.

At least one wall of the cooling means 1 is provided with a plurality of LED components 31 for a UV radiation source. The LED components emit radiation from a wavelength range of for example greater than or equal to 255 nm and less than or equal to 270 nm. LED components with different emission spectra from the UV range are commercially available and are therefore not detailed.

Furthermore at least one wall of the cooling means 1 is provided with LED components 41 for a light source. For example the same wall or the same walls are provided with LED components 41 for a light source, which are provided with LED components 31 for a UV radiation source. The LED components 41 for the light source emit light which is visible to the human eye. These LED components are also commercially available and are known to one skilled in the art so that they are not further explained here.

At least some of the LED components 31, 41 of the light source and of the UV radiation source are for example mounted electrically and mechanically on a common circuit board, see FIG. 4. In one specific example all LED components 31, 41 are mounted electrically and mechanically on a common circuit board 7. The circuit board 7 is located between the wall 9 of the cooling means 1 and the LED components 31, 41.

The LED components 31, 41 are covered for example by means of a cover plate and are separated from the cooling space 2. In this way they are mechanically protected. The cover plate 8 consists of a material which is transparent both to visible light and also to UV radiation. For example the cover pane has silica glass or consists essentially or completely of silica glass.

The cooling means 1 has a switching means which comprises a first switching element 61 and a second switching element 62, see FIG. 1. The switching elements are pressed together and/or inserted into one another when the closing means 5 is closed.

For example, the second switching element 62 comprises two electrodes which are electrically separated from one another and which each are electrically connected to the electrical line of a circuit. See in this respect FIGS. 2a and 2b and FIGS. 3a and 3b, the electrical lines and the other details of the circuit not being shown. Possibilities for execution of a suitable circuit are fundamentally known to one skilled in the art.

The first switching element 61 can be an electrode which is pressed against the second switching element 62 when the closing means 5 is closed and the two electrodes of the second switching element 62 are connected to one another in an electrically conductive manner. In this way the electrical line is closed and causes switching.

According to one embodiment which is illustrated in FIGS. 2a and 2b, the first switching element 61 is made flat and presses from the outside against the second switching element 62.

FIGS. 3a and 3b show another sample configuration of the switching means. Here the first switching element 61 has an electrically conductive projection, while the second switching element 62 has a corresponding depression which the first switching element 61 engages when the closing means 5 is closed. In this way in turn an electrical line is closed and accordingly electrical switching is effected.

The specific possible embodiments of the other components of the switching means and of the trigger circuit for a UV radiation source or light source are generally known to one skilled in the art and are therefore not further detailed here.

The circuit for triggering the LED components 31, 41 for example has a timer switch which ensures that the LED components 31 for the UV radiation source for example after each closing are operated for a defined time and afterwards are automatically turned off.

Alternatively the UV radiation source 3 can also be in operation over a longer time. For example, it can always be continuously operated when the cooling means is closed. In this case the emission output of the LED components 31 for the UV radiation source can be chosen and/or set to be relatively small.

The LED components of the UV radiation source are for example uniformly distributed in the refrigerator so that it is illuminated as uniformly as possible. In this way not only is the circulating air in the cooling space 2 uniformly disinfected, but also surfaces of the cooling means and surfaces of the foods located in the cooling space.

Foods which are contained in commercial packaging and storage materials and are sealed by them are not irradiated by the UV radiation of the UV radiation source since the packaging and storage materials effectively absorb it in general. This ensures that desired bacteria cultures as can be present for example in dairy products such a yogurts cannot be killed.

In addition or alternatively at least some of the LED-components of the UV radiation source can be arranged clustered, for example. In this way for example in certain regions of the cooling space, for example in the vegetable bin, especially strong irradiation with UV radiation can be specifically ensured. Moreover, for LED components which are arranged clustered for example by means of suitable optical means such as scattering lenses relatively uniform illumination of the cooling space can be implemented.

Microorganisms can be reliably killed in the cooling space 2 of the cooling means 1. By eliminating or at least significantly reducing molds, foods, such as especially vegetables, fruit, meat and sausage, keep much longer. This has the additional advantage that a temperature of roughly 7° C. to 8° C. can be sufficient in the refrigerator. The result of this is less energy consumption in operation of the cooling means and thus a reduction of environmental pollution by lower CO² emission. It is for example possible to reduce CO² emission by 10% compared to conventional refrigerators.

The invention is not limited to these embodiments by the description of the invention using them. Rather the invention comprises any new features and any combination of features; this includes especially any combination of features in the claims even if this feature or this combination itself is not explicitly given in the claims or embodiments.

1. Cooling means with a cooling space, characterized in that there is a UV radiation source which is suited to emitting electromagnetic radiation from the ultraviolet region, and in its operation the cooling space is irradiated with this radiation.

2. Cooling means as claimed in claim 1, wherein the cooling means is a refrigerator or a mobile cooler box.

3. Cooling means as claimed in claim 1, wherein the UV radiation source has at least one LED component which is suited to emitting electromagnetic radiation.

4. Cooling means as claimed in claim 3, wherein the UV radiation source has several LED components.

5. Cooling means as claimed in claim 1, wherein the UV radiation source is suited to emitting electromagnetic radiation from a wavelength range of less than or equal to 300 nm.

6. Cooling means as claimed in claim 1, wherein the UV radiation source is suited to emitting electromagnetic radiation from a wavelength range between 250 nm and 270 nm inclusive.

7. Cooling means as claimed in claim 1, wherein a closing means for closing and opening an opening of the cooling space and a switching means are contained, the switching means turning off an electrical trigger circuit for the UV light source as long as the closing means is opened.

8. Cooling means as claimed in claim 1, wherein there is a light source with at least one LED component which is suited to emitting light which is visible to the human eye, and in its operation the cooling space is illuminated and the UV radiation source is turned off.

9. Cooling means as claimed in claim 8, wherein the light source has several LED components.

10. Cooling means as claimed in claim 8, wherein the UV radiation source has at least one LED component, the UV radiation source and the light source having a common circuit board on which the LED components of the UV radiation source and of the light source are electrically and mechanically mounted.

11. Cooling means as claimed in claim 2, wherein the UV radiation source has at least one LED component which is suited to emitting electromagnetic radiation.

12. Cooling means as claimed in claim 2, wherein the UV radiation source is suited to emitting electromagnetic radiation from a wavelength range of less than or equal to 300 nm.

13. Cooling means as claimed in claim 2, wherein the UV radiation source is suited to emitting electromagnetic radiation from a wavelength range between 250 nm and 270 nm inclusive.

14. Cooling means as claimed in claim 2, wherein a closing means for closing and opening an opening of the cooling space and a switching means are contained, the switching means turning off an electrical trigger circuit for the UV light source as long as the closing means is opened.

15. Cooling means as claimed in claim 2, wherein there is a light source with at least one LED component which is suited to emitting light which is visible to the human eye, and in its operation the cooling space is illuminated and the UV radiation source is turned off.

16. Cooling means as claimed in claim 9, wherein the UV radiation source has at least one LED component, the UV radiation source and the light source having a common circuit board on which the LED components of the UV radiation source and of the light source are electrically and mechanically mounted.
